(19) United States
(12) Patent Application Publication
(10) Pub. No.: US 2019/0224373 A1
(43) Pub. Date: Jul. 25, 2019

(54) CHITOSAN-CALCIUM PHOSPHATE COMPOSITE AS ODONTOINDUCTIVE DENTAL FILLINGS AND METHODS OF MAKING AND USING

(71) Applicants: Melissa Krebs, Englewood, CO (US); Matthew Osmond, Golden, CO (US)

(72) Inventors: Melissa Krebs, Englewood, CO (US); Matthew Osmond, Golden, CO (US)

(21) Appl. No.: 16/256,352

(22) Filed: Jan. 24, 2019

Related U.S. Application Data
(60) Provisional application No. 62/621,382, filed on Jan. 24, 2018.

(51) Int. Cl. A61L 27/52; A61L 27/20; A61L 27/12; A61L 27/54; A61K 6/033; A61K 6/00; A61K 6/097

(52) U.S. Cl. CPC A61L 27/52; A61L 27/20; A61L 27/12; A61L 27/54; A61L 2430/02; A61K 6/0067; A61K 6/097; A61L 2300/414; A61K 6/033

(57) ABSTRACT

A dental or bone material comprising a polysaccharide, diepoxide and calcium phosphate. The polysaccharide can be a chitosan, and the polysaccharide is modified to be soluble. The combination of the polysaccharide and diepoxide form a gel, which can be cured to form the implant. The curing does not require equipment, but instead the material is self-curing. Methods of making and using the material are also disclosed.

### CROSS REFERENCE TO RELATED APPLICATION

This application claims priority and the benefit under 35 U.S.C. § 119(e) to U.S. Provisional Patent Application Ser. No. 62/621,382 filed Jan. 24, 2018, which is incorporated herein in its entirety by reference.

### FIELD OF THE INVENTION

The present invention relates to dental and bone implant materials, methods of making and methods of using the same.

### BACKGROUND

Dental caries or tooth decay is the most frequent but treatable oral disease. Current materials provide immediate restoration of function, but do not provide a platform for dental tissue or bone regeneration. For example, in tooth repair, silver amalgam or gold fillings restore function but have no effect on tissue regeneration and have limited lifespan. These colored materials also stand out compared to the other teeth of the patient. White composite fillings also restore function and lack tissue regeneration and also have limited lifespan of about 15 years. These materials might also require additional equipment to cure the material, which can be difficult to use in the confined space of a patient's mouth. Alternative filling materials also have a variety of handling properties, some of which can be difficult to administer.

### SUMMARY

The chitosan-calcium phosphate materials of the present invention restore function to the tooth after decay caused by dental caries by providing an injectable, mechanically stable, and regenerative filling. The present invention can be particularly suited for clinical deployment. In some embodiments, a premixed solution (containing for example the chitosan in the water) can be provided to a user in a syringe with a setting agent (for example the diepoxide). While other methods of application can be used with the present invention, an injectable application provides ease of use and control over the gelation time of the material. In addition, this product can alternatively be used in a variety of hard tissue applications from bone adhesive to bone fillers.

A component of the material of the present invention is a modified polysaccharide, for example a modified chitosan. An example of a suitable modified chitosan is O-carboxymethyl-chitosan. Chitosan is useful for its inherent chemical properties as well as its biocompatibility. Chitosan is also an acid-soluble material that is not often able to be used in water-based formulations. However, the present invention utilizes a modified chitosan, which, in one embodiment of the present invention, has been modified with a carboxymethyl group thereby enabling solubility of the polymer material in aqueous mediums.

After the chitosan is modified, it can be reacted with a diepoxide. The modified chitosan and the diepoxide linking agent results in the formation of an insoluble gel matrix. The gel matrix includes interconnected chitosan molecules with high water content (greater than about 85 wt. % water in the gel, in some embodiments between about 85 wt. % and about 98 wt. %, in some embodiments between about 87 wt. % and about 95 wt. %). FIG. 1 illustrates the reaction of the modified polysaccharide with a diepoxide to form a crosslinked matrix. Advantageously, the material of the present invention is self-setting or self-curing and does not require the use of additional equipment to cure the material. Furthermore, cure depth is uniform throughout.

The hydrogel can include calcium phosphate (CaP). The CaP in the hydrogel can adhere to native CaP on the surface of the bone or tooth. The addition of CaP microparticles in the gel can provide the implantable material with mechanical stability as well as a biocompatible surface that can support cell growth and guide differentiation. Additionally, the material of the present invention can provide an immediate restorative filling with long term regenerative properties.

The present invention can provide a gelation time of less than about 30 minutes, with a storage modulus of between about 100 kPa and about 2.5 MPa in some embodiments. The material of the present invention is also biocompatible. Cell survival can exceed initial seeding and proceed for up to about 3 weeks, in some embodiments about 4 weeks, with markers of odonto/osteogenesis being expressed. This material can improve on current dental filling composites by providing a method for cellular integration and regeneration of dental tissue damaged by cavities.

The present invention has other advantages over current fillings. The materials of the present invention include injectable hydrogel composites with tunable mechanical properties and biomimetic cell environment. The hydrogels can be tunable based on the amount of diepoxide and/or additive included in the hydrogel.

The mixing and crosslinking process of the present invention is analogous to common household formulations of two-part epoxy resin glues, where the contact of the two components forms an injectable gel that then cures to a hardened material. For practical use, the cure time and hardness can be selectively determined as illustrated in various examples herewith.

An advantage of the present invention is that the formulation of composite does not require the use of organic solvents or silica. The incorporation of the silica filler can reinforce the polymeric resin and improves the ultimate mechanical properties of the permanent filling. Even without this silica filler, the formulation of a regenerative implantable materials of the present invention meets at least some of the existing standards, including standards set forth in ISO 4049 (fourth edition from Oct. 1, 2009, which is incorporated by reference herewith. For example, materials of the present invention fall within the water sorption and solubility standards set forth in ISO 4049 (fourth edition). Furthermore, CaP materials exhibit positive effects towards improving material bioactivity, and also enhance mechanical robustness of composite materials.

The CaP utilized with the present invention can be hydroxyapatite (HA), or other CaP materials, and can be various shapes, such as nanorods, whiskers, particles, and combinations thereof, and uniform in size. Combinations of different CaP materials can also be used without deviating from the present invention. The size and shape of the HA material can provide easy incorporation into the materials, while maintaining injectability of the material and optimizing mechanical strength. In some embodiments, the incorporation of the CaP-nanorods can result in a significant increase in the mechanical strength of the materials as illustrated in FIG. 2. When the HA is not a nanorod, but is instead a different shape, similar or increased strengths also result. FIG. 2 illustrates the advantage of incorporation of the HA as compared to materials not incorporating the CaP. The chitosan hydrogels formulated without CaP-nanorods (no rods) exhibit mechanical strength of about 10 kPa. The incorporation of 30 wt. % CaP into the hydrogel matrix resulted in a significant increase in hydrogel strength to about 300 kPa. Further addition of CaP to 40 wt. % resulted in a further increase in mechanical strength to about 600 kPa. In other words, the present invention can impart significant improvements to the mechanical strength by modifying the amount of CaP into the hydrogel matrix. This advantage is particularly important for fillings that require both strength during restoration and regeneration, for example during temporary tooth restoration.

An aspect of the invention is an implantable material. The implantable material comprises a cured modified polysaccharide crosslinked with a diepoxide. The modified polysaccharide can include a modified chitosan (where the chitosan has been modified to be soluble in an aqueous solution). The diepoxide can be 1,4 butanediol diglycidyl ether or any material containing multiple glycidyl groups, or combinations of diepoxides.

An aspect of the invention is a method of forming a hydrogel. The method includes providing a modified polysaccharide, where the modified polysaccharide comprises a chitosan modified to form a soluble chitosan. The modified polysaccharide is then crosslinked with a diepoxide to form the hydrogel.

An aspect of the invention is a method to form an implantable material. The method includes providing between about 2 wt. % and about 15 wt. % of a modified polysaccharide. The modified polysaccharide comprises a polysaccharide modified with a carboxymethyl. Between about 5 wt. % and about 20 wt. % of an diepoxide and between about 0 wt. % and about 40 wt. % of a calcium phosphate are mixed with the polysaccharide to form the implantable material.

An aspect of the invention is a void filling gel material. The material comprises a crosslinked modified polysaccharide, and a diepoxide. The gel hardens to form an implantable material.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates a schematic for modified polysaccharide crosslinking with diepoxide groups;

FIG. 2 illustrates the storage modulus for the implant materials of the present invention as a function of the strain for various compositions of the present invention;

FIG. 3 illustrates Fourier transform infrared (FTIR) spectra of chitosan and carboxymethyl chitosan;

FIG. 4 illustrates HA whiskers, and composite materials with characteristic peaks labeled with dotted lines;

FIG. 5 illustrates X-ray diffraction pattern of the HA whiskers illustrating the crystallinity and identity of hydroxyapatite present;

FIG. 6 illustrates a scanning electron microscope (SEM) image of HA whiskers;

FIG. 7 illustrates a SEM image of a composite material of the present invention with 30 wt. % HA whiskers;

FIG. 8 illustrates a SEM image of a composite material of the present invention with 40 wt. % HA whiskers;

FIG. 9 illustrates a storage modulus graft of three composite materials of the present invention with varying amounts of HA whisker loading;

FIG. 10A illustrates the swelling ratio of three composite materials of the present invention with varying amounts of HA whisker loading;

FIG. 10B illustrates the degradation percentage of three composite materials of the present invention with varying amounts of HA whisker loading;

FIG. 11 illustrates the relative number of cells for composite materials of the present invention containing varying amounts of HA whisker loadings over a three week period;

FIG. 12 illustrates the relative expression of RUNX2 for composite materials of the present invention comprising 0 wt. %, 30 wt. % and 40 wt. % HA whisker at two or three weeks;

FIG. 13A illustrates a confocal micrograft of dental pulp stem cells on the surface of a composite material of the present invention that includes 0 wt. % HA whiskers after 14 days;

FIG. 13B illustrates a confocal micrograft of dental pulp stem cells on the surface of a composite material of the present invention that includes 0 wt. % HA whiskers after 21 days;

FIG. 13C illustrates a confocal micrograft of dental pulp stem cells on the surface of a composite material of the present invention that includes 20 wt. % HA whiskers after 14 days;

FIG. 13D illustrates a confocal micrograft of dental pulp stem cells on the surface of a composite material of the present invention that includes 20 wt. % HA whiskers after 21 days;

FIG. 13E illustrates a confocal micrograft of dental pulp stem cells on the surface of a composite material of the present invention that includes 40 wt. % HA whiskers after 14 days;

FIG. 13F illustrates a confocal micrograft of dental pulp stem cells on the surface of a composite material of the present invention that includes 40 wt. % HA whiskers after 21 days;

FIG. 14A illustrates a transmission electron micrograft (TEM) showing DCPD;

FIG. 14B illustrates a TEM showing DCPD/TEG;

FIG. 14C illustrates a TEM showing HA;

FIG. 14D illustrates a TEM showing HA/TEG;

FIG. 15 illustrates the storage modulus of the three composite materials of the present invention with 0 wt. %, 20 wt. % and 40 wt. % loading of the four calcium phosphate particles;

FIG. 16 illustrates the relative number of dental pulp stem cells over a three week period for the 0 wt. %, 20 wt. % and 40 wt. % loaded; and

FIG. 17 illustrates BSA release profiles for four types of composite materials of the present invention over four weeks.

### DETAILED DESCRIPTION

The present invention relates to a water based defect filing material that exhibits self-curing technology. The material can be particularly useful for dental or bone fillings or adhesives. The material is a polymeric material formulated as a hydrogel. The hydrogel is a water-based insoluble membrane that can support cellular activity, which allows for enhanced biological compatibility of the material as compared to acrylic resins that require organic solvents. The present invention also includes methods to form the material or precursor materials and methods of using the material, the material in the pre-cured state, as well as the material once cured.

An aspect of the present invention is a method of forming a hydrogel. The method includes combining a modified polysaccharide with a diepoxide. The modified polysaccharide can include a chitosan and a carboxymethyl group.

The modified polysaccharide can be in the form of a solid, which can be dissolved in an aqueous fluid prior to mixture with the diepoxide. Between about 2 wt. % and about 15 wt. % of the modified polysaccharide can be dissolved in between about 85 wt. % and about 98 wt. % of the aqueous fluid. In some embodiments, the aqueous fluid can be water (e.g. tap, distilled, deionized, etc.), a buffered saline (e.g. phosphate buffered saline), or combinations thereof. Advantageously, the aqueous fluid is not an organic solvent such as chloroform, xylene or acetone.

The modified polysaccharide is crosslinked with a diepoxide. In some embodiments, the mass ratio of the modified polysaccharide to the diepoxide can be between about 1:200 and about 1:50.

The hydrogel can include at least one additive. Suitable additives include CaP, growth factors, chemokines (e.g. SDF-1a), a bovine serum additive, and combinations thereof. The additive can be included in the aqueous solution comprising the modified polysaccharide, the diepoxide or added separately from the modified polysaccharide and the diepoxide. However, the additive can be more evenly distributed if it is added to the aqueous modified polysaccharide. Thus, in some embodiments, the modified polysaccharide solution can further include additive, where the weight percent of the additives can be between about 0 wt. % and about 40 wt. % of the total weight of the composite. The hydrogel can include between about 0 wt. % and about 40 wt. % of the total weight of the additive. The mass ratio of the modified polysaccharide to the diepoxide to the additive can be between about 1:1:0 and about 1:1:40.

When the additive is CaP, the CaP can be HA, for example HA whiskers, HA particles, or combinations thereof, dicalcium phosphate (DCPD) particles or rods, and combinations thereof. In some embodiments, tetraethylene glycol diacrylate (TEG) can be added to the CaP to prevent agglomeration. Thus, additional suitable additives can include TEG modified HA particles, TEG modified DCPD particles and combinations thereof, which can be also combined with one or more of HA whiskers, HA particles, or DCPD particles. Excess TEG can be provided to the CaP, which can later be purified to remove excess TEG remaining in the aqueous solution comprising the modified polysaccharide, additive and TEG, or the gel.

The additive, for example CaP, can be provided in different shapes and sizes, and can be chosen to be suitable for injection. In some embodiments, the additive can be nanorods, nano particles, nano sheets, and combinations thereof. The diameter or thickness of additive can be between about 2 microns and about 7 microns, a length and/or width of between about 2 microns and about 85 microns, in some embodiments between about 25 microns and about 85 microns.

The diepoxide can be 1,4 butanediol diglycidyl ether or any material containing multiple glycidyl groups, or combinations of diepoxides. The modified polysaccharide can be a modified chitosan. Suitable modifications to chitosan can include carboxymethylation or any modification that increases solubility while leaving the amine groups intact. Incidental materials can be inherently included in the mixture without deviating from the invention. Such incidental materials can include unrefined chitosan, unmodified chitosan, unreacted modification precursor, unreacted calcium phosphate precursors, or combinations thereof. Between about 0 wt. % and 1 wt. % of the incidental materials can be included in the hydrogel.

An aspect of the invention is a hydrogel. The hydrogel can be a modified polysaccharide crosslinked with a diepoxide. The modified polysaccharide can be present in the hydrogel at a weight percentage of between about 2 and about 15, with the balance being the diepoxide and up to about 1 wt. % of incidental materials.

The modulus of the hydrogel can be between about 1000 Pa and about 2.5 MPa. The swelling ratio of the hydrogel can be between about 1 and about 11. The degradation can be between about 0 and about 40% over a time period of about 0 to about 7 days. The relative number of cells for the hydrogels can be between about 0.5 ng DNA/mL and about 15 ng DNA/mL over a period of three weeks. The fold change of the hydrogel, which can be the relative expression of RUNX2, can be between about 1 and about 2.5 over a period of about 14 days and about 21 days. Furthermore, in some embodiments, dental pulp stem cells in the surface of the hydrogel can be present after 7 days, and up to at least about 21 days. The compressive modulus of the hydrogel can be between about 100 kPa and about 3000 kPa. In embodiments where a releasing additive is incorporated into the hydrogel (e.g. BSA), between about 0 micrograms and about 2500 micrograms of the additive can be released over a time period, in some embodiments with the time period being up to about 30 days.

The modified polysaccharide can be in the form of a solid, which can be dissolved in an aqueous fluid prior to mixture with the diepoxide. Between about 2 wt. % and about 15 wt. % of the modified polysaccharide can be dissolved in between about 85 wt. % and about 98 wt. % of the aqueous fluid. In some embodiments, the aqueous fluid can be water (e.g. tap, distilled, deionized, etc.), buffered saline (e.g. phosphate buffered saline), or combinations thereof. Advantageously, the aqueous fluid is not an organic solvent such as chloroform, xylene or acetone.

In some embodiments, the mass ratio of the modified polysaccharide to the diepoxide can be between about 1:200 and about 1:50. The hydrogel can include at least one additive. Suitable additives include CaP, growth factors, chemokines (e.g. SDF-1a), a bovine serum additive, and combinations thereof. When the additive is included in the hydrogel, it can be homogenously distributed throughout the polymer. However, in some embodiments, the additive can be agglomerated or concentrated at multiple locations within the polymer hydrogel. Thus, in some embodiments, the hydrogel can include additives, where a total weight percent of the additive being between about 0 wt. % and about 40 wt. % of the total weight of the hydrogel, with between about 2 wt. % and 15 wt. % of the modified polysaccharide, up to about 1 wt. % of incidental materials, and the balance being the diepoxide. The mass ratio of the modified polysaccharide to the diepoxide to the additive can be between about 1:1:0 and about 1:1:40.

When the additive is CaP, the CaP can be HA, for example HA whiskers, HA particles, or combinations thereof, DCPD particles or rods, and combinations thereof. In some embodiments, TEG can be added to the CaP to prevent agglomeration. Thus, additional suitable additives can include TEG modified HA particles, TEG modified DCPD particles and combinations thereof, which can be also combined with one or more of HA whiskers, HA particles, or DCPD particles. Excess TEG can be provided to the CaP during the production of the hydrogel, which can be removed from the hydrogel.

The additive, for example CaP, can be provided in different shapes and sizes, and can be chosen to be suitable for injection. In some embodiments, the additive can be nanorods, nano particles, nano sheets, and combinations thereof. The diameter or thickness of additive can be between about 2 microns and about 7 microns, a length and/or width of between about 2 microns and about 85 microns, in some embodiments between about 25 microns and about 85 microns.

The diepoxide can be 1,4 butanediol diglycidyl ether or any material containing multiple glycidyl groups, or combinations of diepoxides. The modified polysaccharide can be a modified chitosan. Suitable modifications to chitosan can include carboxymethylation or any modification that increases solubility while leaving the amine groups intact. Incidental materials can be inherently included in the mixture without deviating from the invention. Such incidental materials can include unrefined chitosan, unmodified chitosan, unreacted modification precursor, or unreacted calcium phosphate precursors, or combinations thereof. Between about 0 wt. % and 1 wt. % of the incidental materials can be included in the hydrogel.

An aspect of the invention is a method to form an implantable material. The method includes mixing between about 2 wt. % and about 15 wt. % of a modified polysaccharide, between about 5 wt. % and about 20 wt. % of an diepoxide, and between about 0 wt. % and about 40 wt. % of a CaP to form the implantable material. The modified polysaccharide can include a chitosan modified with a carboxymethyl group.

The modified polysaccharide can be in the form of a solid, which can be dissolved in an aqueous fluid prior to mixture with the diepoxide. The modified polysaccharide can be dissolved in between about 85 wt. % and about 98 wt. % of the aqueous fluid. In some embodiments, the aqueous fluid can be water (e.g. tap, distilled, deionized, etc.), buffered saline (e.g. phosphate buffered saline), or combinations thereof. Advantageously, the aqueous fluid is not an organic solvent such as chloroform, xylene or acetone.

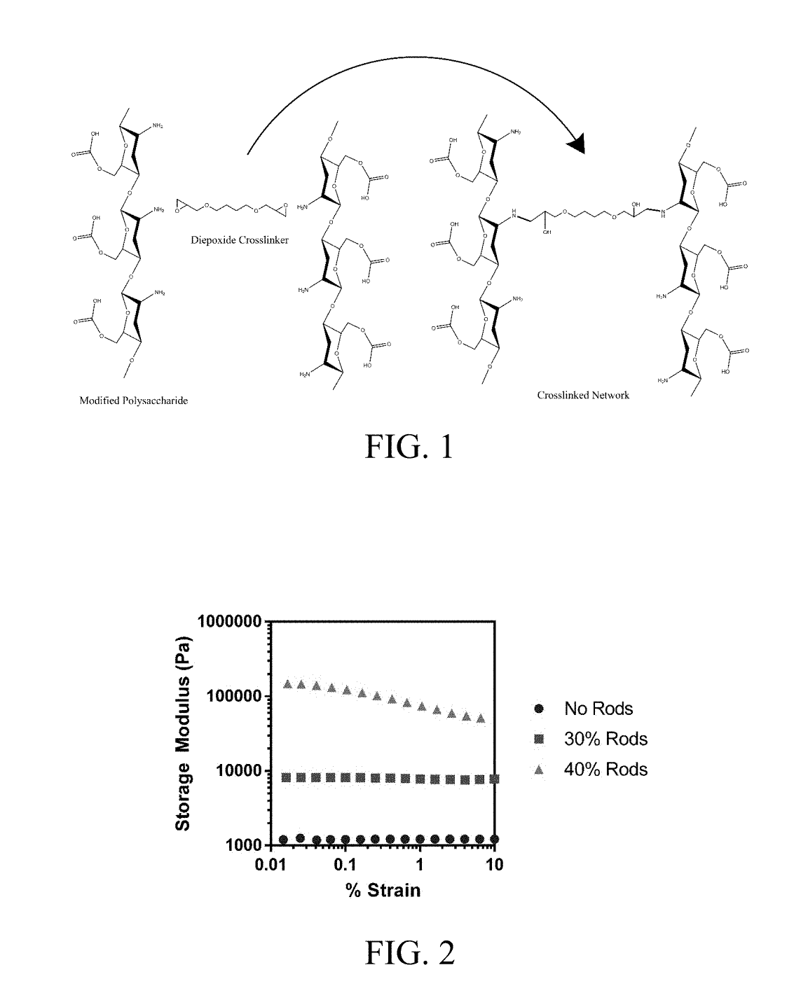

The modified polysaccharide can be a modified chitosan comprising an amino radical (NH₂) for reaction with the diepoxide. FIG. 1 illustrates an example of the hydrogel formed by the present invention. The modified polysaccharide is crosslinked with a diepoxide. In some embodiments, the mass ratio of the modified polysaccharide to the diepoxide can be between about 1:200 and about 1:50.

The hydrogel can include at least one additive. Suitable additives include CaP, growth factors, chemokines (e.g. SDF-1a), a bovine serum additive, and combinations thereof. The additive can be evenly distributed in the hydrogel or can be agglomerated. The total weight percentage of all additives in the hydrogel can be between about 0 wt. % and about 40 wt. %. Thus, the mixture can include between about 2 wt. % and about 15 wt. % of a modified polysaccharide, between about 5 wt. % and about 20 wt. % of an diepoxide, between about 0 wt. % and about 40 wt. % of CaP and any other additives, and up to 1 wt. % of incidental materials to equal a total of 100 wt. % of the components in the hydrogel. In some embodiments, the mass ratio of the modified polysaccharide to the diepoxide to the additive(s) can be between about 1:1:0 and about 1:1:40.

The CaP can be HA, for example HA whiskers, HA particles, or combinations thereof, DCPD particles or rods, and combinations thereof. In some embodiments, TEG can be added to the CaP to prevent agglomeration. Thus, additional suitable additives can include TEG modified HA particles, TEG modified DCPD particles and combinations thereof, which can be also combined with one or more of HA whiskers, HA particles, or DCPD particles. Excess TEG can be removed from the hydrogel.

The additive, for example CaP, can be provided in different shapes and sizes, and can be chosen to be suitable for injection. In some embodiments, the additive can be nanorods, nano particles, nano sheets, and combinations thereof. The diameter or thickness of additive can be between about 2 microns and about 7 microns, a length and/or width of between about 2 microns and about 80 microns, in some embodiments between about 25 microns and about 85 microns.

The diepoxide can be 1,4 butanediol diglycidyl ether or any material containing multiple glycidyl groups, or combinations of diepoxides. The modified polysaccharide can be a modified chitosan. Suitable modifications to chitosan can include carboxymethylation or any modification that increases solubility while leaving the amine groups intact. Incidental materials can be inherently included in the mixture without deviating from the invention. Such incidental materials can include unrefined chitosan, unmodified chitosan, unreacted modification precursor, or unreacted calcium phosphate precursors, or combinations thereof. Between about 0 wt. % and 1 wt. % of the incidental materials can be included in the hydrogel.

The implantable material can be in the form of a gel. After application to a defect, the implantable material can be cured to form the implant. The curing of the implantable material does not require additional equipment, for example an ultraviolet light, elevated heat exposure, ultrasound or combinations thereof. Nor does the reaction to form the implant require the use of catalysts in the gel. Rather, the implantable material of the present invention can be self-curing. The implantable material can be cured for between about 10 minutes and about 40 minutes at a temperature of between about 25° C. and about 40° C. to form the implant material. This is not to say that additional equipment or catalysts cannot be used in the curing process. Rather, it should be understood that the reaction does not require additional equipment or catalysts, and in some embodiments additional equipment or catalysts are not used to cure the material.

The implantable material can be provided in a kit to a user. The kit can include the modified polysaccharide and the diepoxide for mixing by the end user. In some embodiments, the materials can be provided by way of a multiple part or duel part mixture that accounts for the mixing ratio of the component parts. In some embodiments, the modified polysaccharide can include any additives required for the mixture.

The implantable material can be used as a dental implant or a bone implant. The material can be applied to a void or defect in a patient, such as a cavity or a bone void, by injection, painting, filing, or combinations thereof.

An aspect of the invention is a biocompatible gel material. The material includes a modified polysaccharide, and a diepoxide. The gel hardens to form the biocompatible implantable material.

The modulus of the gel can be between about 1000 Pa and about 2.5 MPa. The swelling ratio of the gel can be between about 1 and about 11. The degradation can be between about 0% and about 40% over a time period of about 0 to about 7 days. The relative number of cells for the gels can be between about 0.5 ng DNA/mL and about 15 ng DNA/mL over a period of three weeks. The fold change of the gel, which can be the relative expression of RUNX2, can be between about 1 and about 2.5 over a period of about 14 days and about 21 days. Furthermore, in some embodiments, dental pulp stem cells in the surface of the gel can be present after 7 days, and up to at least about 21 days. The compressive modulus of the gel can be between about 100 kPa and about 3000 kPa. In embodiments where a releasing additive is incorporated into the gel (e.g. BSA), between about 0 micrograms and about 2500 micrograms of the additive can be released over a time period, in some embodiments with the time period being up to about 30 days.

The modified polysaccharide can be in the form of a solid, which can be dissolved in an aqueous fluid prior to mixture with the diepoxide. Between about 2 wt. % and about 15 wt. % of the modified polysaccharide can be dissolved in between about 85 wt. % and about 98 wt. % of the aqueous fluid. In some embodiments, the aqueous fluid can be water (e.g. tap, distilled, deionized, etc.), buffered saline (e.g. phosphate buffered saline), or combinations thereof. Advantageously, the aqueous fluid is not an organic solvent such as chloroform, xylene or acetone.

In some embodiments, the mass ratio of the modified polysaccharide to the diepoxide can be between about 1:200 and about 1:50. The gel can include at least one additive. Suitable additives include CaP, growth factors, chemokines (e.g. SDF-1a), a bovine serum additive, and combinations thereof. When the additive is included in the gel, it can be homogenously distributed throughout the polymer. However, in some embodiments, the additive can be agglomerated or concentrated at multiple locations within the polymer gel. Thus, in some embodiments, the gel can include additives, where a total weight percent of the additive being between about 0 wt. % and about 40 wt. % of the total weight of the gel, with between about 2 wt. % and 15 wt. % of the modified polysaccharide, up to about 1 wt. % of incidental materials, and the balance being the diepoxide. The mass ratio of the modified polysaccharide to the diepoxide to the additive can be between about 1:1:0 and about 1:1:40.

When the additive is CaP, the CaP can be HA, for example HA whiskers, HA particles, or combinations thereof, DCPD particles or rods, and combinations thereof. In some embodiments, TEG can be added to the CaP to prevent agglomeration. Thus, additional suitable additives can include TEG modified HA particles, TEG modified DCPD particles and combinations thereof, which can be also combined with one or more of HA whiskers, HA particles, or DCPD particles. Excess TEG can be provided to the CaP during the production of the gel, which can be removed from the gel.

The additive, for example CaP, can be provided in different shapes and sizes, and can be chosen to be suitable for injection. In some embodiments, the additive can be nanorods, nano particles, nano sheets, and combinations thereof. The diameter or thickness of additive can be between about 2 microns and about 7 microns, a length and/or width of between about 2 microns and about 85 microns, in some embodiments between about 25 microns and about 85 microns.

The diepoxide can be 1,4 butanediol diglycidyl ether or any material containing multiple glycidyl groups, or combinations of diepoxides. The modified polysaccharide can be a modified chitosan. Suitable modifications to chitosan can include carboxymethylation or any modification that increases solubility while leaving the amine groups intact. Incidental materials can be inherently included in the mixture without deviating from the invention. Such incidental materials can include unrefined chitosan, unmodified chitosan, unreacted modification precursor, or unreacted calcium phosphate precursors, or combinations thereof. Between about 0 wt. % and 1 wt. % of the incidental materials can be included in the gel.

An aspect of the invention is a method of repairing a void in a patient. A user mixes the modified chitosan, and a diepoxide linking agent to form a gel. The mixing ratio of the modified polysaccharide to the diepoxide is between about 5:1 and about 1:1. The gel is applied to the void of a patient before the gel cures, typically within about 40 minutes of mixing the modified polysaccharide and the diepoxide linking agent. The gel is then cured to form the dental implant.

The modulus of the gel can be between about 1000 Pa and about 2.5 MPa. The swelling ratio of the gel can be between about 1 and about 11. The degradation can be between about 0% and about 40% over a time period of about 0 to about 7 days. The relative number of cells for the gels can be between about 0.5 ng DNA/mL and about 15 ng DNA/mL over a period of three weeks. The fold change of the gel, which can be the relative expression of RUNX2, can be between about 1 and about 2.5 over a period of about 14 days and about 21 days. Furthermore, in some embodiments, dental pulp stem cells in the surface of the gel can be present after 7 days, and up to at least about 21 days. The compressive modulus of the gel can be between about 100 kPa and about 3000 kPa. In embodiments where a releasing additive is incorporated into the gel (e.g. BSA), between about 0 micrograms and about 2500 micrograms of the additive can be released over a time period, in some embodiments with the time period being up to about 30 days.

The modified polysaccharide can be in the form of a solid, which can be dissolved in an aqueous fluid prior to mixture with the diepoxide. Between about 2 wt. % and about 15 wt. % of the modified polysaccharide can be dissolved in between about 85 wt. % and about 98 wt. % of the aqueous fluid. In some embodiments, the aqueous fluid can be water (e.g. tap, distilled, deionized, etc.), buffered saline (e.g. phosphate buffered saline), or combinations thereof. Advantageously, the aqueous fluid is not an organic solvent such as chloroform, xylene or acetone.

In some embodiments, the mass ratio of the modified polysaccharide to the diepoxide can be between about 1:200 and about 1:50. The gel can include at least one additive. Suitable additives include CaP, growth factors, chemokines (e.g. SDF-1a), a bovine serum additive, and combinations thereof. When the additive is included in the gel, it can be homogenously distributed throughout the polymer. However, in some embodiments, the additive can be agglomerated or concentrated at multiple locations within the polymer gel. Thus, in some embodiments, the gel can include additives, where a total weight percent of the additive being between about 0 wt. % and about 40 wt. % of the total weight of the gel, with between about 2 wt. % and 15 wt. % of the modified polysaccharide, up to about 1 wt. % of incidental materials, and the balance being the diepoxide. The mass ratio of the modified polysaccharide to the diepoxide to the additive can be between about 1:1:0 and about 1:1:40.

When the additive is CaP, the CaP can be HA, for example HA whiskers, HA particles, or combinations thereof, DCPD particles or rods, and combinations thereof. In some embodiments, TEG can be added to the CaP to prevent agglomeration. Thus, additional suitable additives can include TEG modified HA particles, TEG modified DCPD particles and combinations thereof, which can be also combined with one or more of HA whiskers, HA particles, or DCPD particles. Excess TEG can be provided to the CaP during the production of the gel, which can be removed from the gel.

The additive, for example CaP, can be provided in different shapes and sizes, and can be chosen to be suitable for injection. In some embodiments, the additive can be nanorods, nano particles, nano sheets, and combinations thereof. The diameter or thickness of additive can be between about 2 microns and about 7 microns, a length and/or width of between about 2 microns and about 85 microns, in some embodiments between about 25 microns and about 85 microns.

The diepoxide can be 1,4 butanediol diglycidyl ether or any material containing multiple glycidyl groups, or combinations of diepoxides. The modified polysaccharide can be a modified chitosan. Suitable modifications to chitosan can include carboxymethylation or any modification that increases solubility while leaving the amine groups intact. Incidental materials can be inherently included in the mixture without deviating from the invention. Such incidental materials can include unrefined chitosan, unmodified chitosan, unreacted modification precursor, or unreacted calcium phosphate precursors, or combinations thereof. Between about 0 wt. % and 1 wt. % of the incidental materials can be included in the gel.

The curing time of the gel can be between about 10 minutes and about 40 minutes. The curing of the gel does not require additional equipment, for example a ultraviolet light, elevated heat exposure, ultrasound or combinations thereof. Nor does the reaction to form the implant require the use of catalysts in the gel. Rather, the gel of the present invention can be self-curing. The gel can be cured for between about 10 minutes and about 40 minutes at a temperature of between about 25° C. and about 40° C. to form the implant material. This is not to say that additional equipment or catalysts cannot be used in the curing process. Rather, it should be understood that the reaction does not require additional equipment or catalysts, and in some embodiments additional equipment or catalysts are not used to cure the material.

Table 1 illustrates the gel time for hydrogels of the present invention with varying amounts of CaP. All values in Table 1 are approximate.

| CaP (wt. %) | Gelation time (minutes) |
|---|---|
| 0 | 32 |
| 30 | 19 |
| 40 | <2 |

An aspect of the invention is an implant. The implant includes a modified polysaccharide that is crosslinked with a diepoxide. The polymer can include an additive.

The implant can include at least one additive. Suitable additives include CaP, growth factors, chemokines (e.g. SDF-1a), a bovine serum additive, and combinations thereof. When the additive is included in the implant, it can be homogenously distributed throughout the implant. However, in some embodiments, the additive can be agglomerated or concentrated at multiple locations within the implant. Thus, in some embodiments, the implant can include additives, where a total weight percent of the additive being between about 0 wt. % and about 40 wt. % of the total weight of the implant, and up to about 1 wt. % of incidental materials, with the balance being the implant material (i.e. crosslinked modified polysaccharide and diepoxide). The modified chitosan can be a chitosan modified with a carboxyl group or modified to be soluble while maintaining the amino group, and the diepoxide can be 1,4 butanediol diglycidyl ether or any material containing multiple glycidyl groups, or combinations of diepoxides. Incidental materials can be inherently included in the mixture without deviating from the invention. Such incidental materials can include unrefined chitosan, unmodified chitosan, unreacted modification precursor, or unreacted calcium phosphate precursors, or combinations thereof. Between about 0 wt. % and 1 wt. % of the incidental materials can be included in the implant.

When the additive is CaP, the CaP can be HA, for example HA whiskers, HA particles, or combinations thereof, DCPD particles or rods, and combinations thereof. In some embodiments, TEG can be added to the CaP to prevent agglomeration. Thus, additional suitable additives can include TEG modified HA particles, TEG modified DCPD particles and combinations thereof, which can be also combined with one or more of HA whiskers, HA particles, or DCPD particles. Excess TEG can be provided to the CaP during the production of the gel, which can be removed from the gel. Thus, in some embodiments, the implant does not include excess TEG.

The additive, for example CaP, can be provided in different shapes and sizes, and can be chosen to be suitable for injection. In some embodiments, the additive can be nanorods, nano particles, nano sheets, and combinations thereof. The diameter or thickness of additive can be between about 2 microns and about 7 microns, a length and/or width of between about 2 microns and about 85 microns, in some embodiments between about 25 microns and about 85 microns.

The implant can be used in a void on a patient. The void can be a dental void such as a cavity, or a bone void. The implant is biocompatible and can promote regeneration in the bone.

Evidence of bone regeneration can be evident using several techniques, or combinations of techniques. For example, bone regeneration can be determined by gene expression, such as RUNX2, osteocalcin, osteopontin, alkaline phosphatase, or other gene expressions. Bone regeneration can also be determined by calcium deposition of cells, visualization repair (e.g. x-ray or micro-CT), or the like.

### EXAMPLES

### Formation of the Materials

Materials of the present invention were formed with varying amounts of calcium phosphate in the hydrogel, where the hydrogel comprises a modified chitosan (chitosan modified with carboxymethyl group) and a diepoxide. In some of the examples, the calcium phosphate added to the hydrogel is in the form of HA rods (“HA whiskers”). Some of the examples utilize DCPD as the calcium phosphate material.

### Example 1: FTIR and X-Ray Diffraction

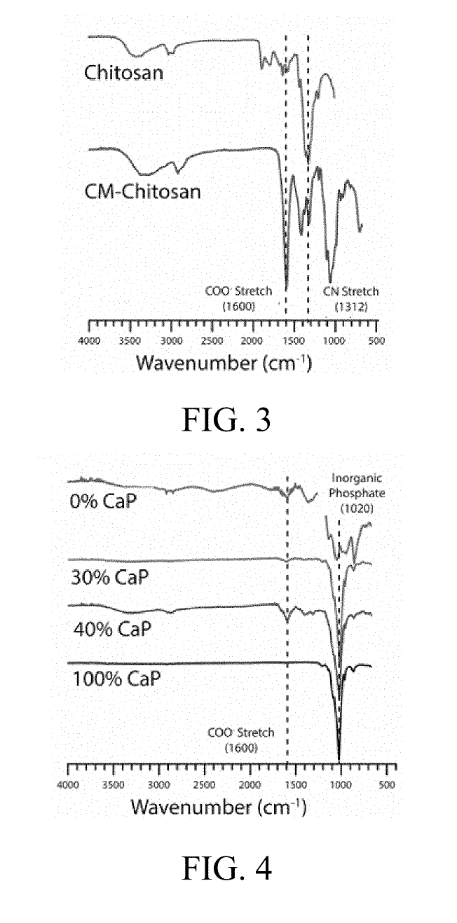

FTIR scans of the present invention were performed to compare the modified chitosan to non-modified chitosan. FIG. 3 illustrates FTIR spectra of chitosan and carboxymethyl chitosan. The COO— stretch at 1600 cm⁻¹ is present in the modified chitosan where it is not present in the chitosan indicating the modification of the chitosan with the carboxymethyl group. The CN stretch is also present in the modified chitosan and not present in the chitosan around 1312 cm⁻¹.

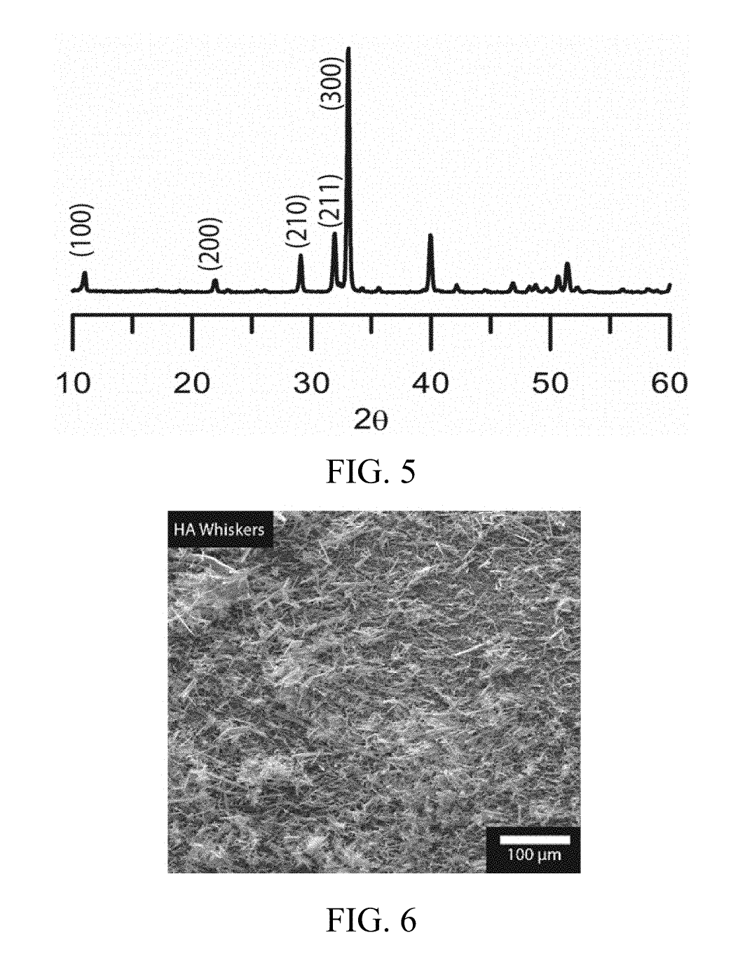

FIG. 4 illustrates modified chitosan with varying amounts of CaP. The control (i.e. 100% CaP) includes only the HA whiskers, where 0% CaP includes only the modified chitosan. The 30% CaP sample and the 40% CaP sample each include an inorganic phosphate signal around 1020 cm⁻¹ and the CN stretch around 1312 cm. FIG. 5 illustrates X-ray diffraction patterns of the HA whiskers illustrating the crystallinity and identity of hydroxyapatite present.

### Example 2: SEM

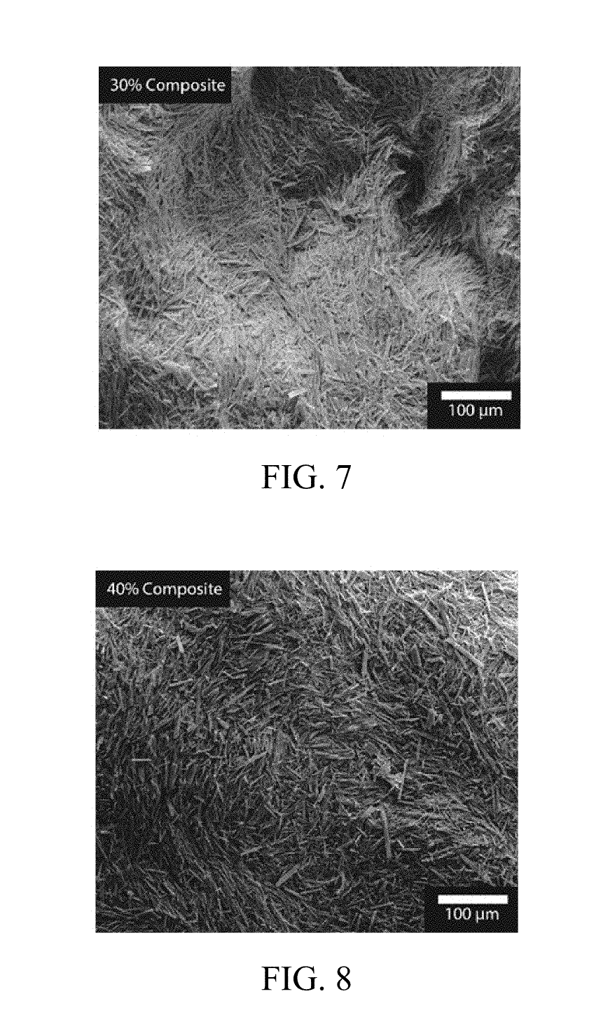

Scanning electron microscope images were taken of HA whiskers, and various compositions of the present invention incorporating the HA whiskers are illustrated in FIGS. 6-8. FIGS. 7 and 8 illustrate that the whiskers are present in the composite after the polymer is formed.

### Example 3: Storage Modulus

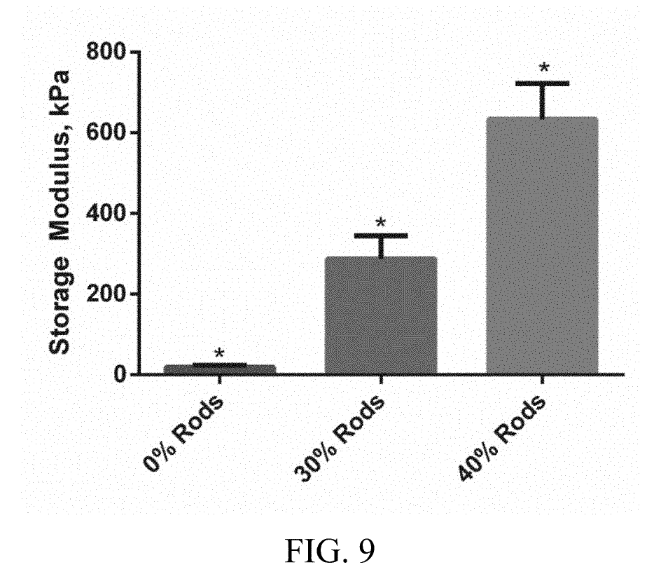

The storage modulus of the three composite materials of the present invention with 0 wt. %, 30 wt. % and 40 wt. % HA whisker loading are illustrated in FIG. 9. The deviation for the materials are significantly different and denoted with a * (p<0.05). FIG. 9 illustrates that the addition of HA, in this illustration HA whiskers, result in higher strength materials compared to when no HA is incorporated into the material.

### Example 4: Swelling Ratio and Degradation

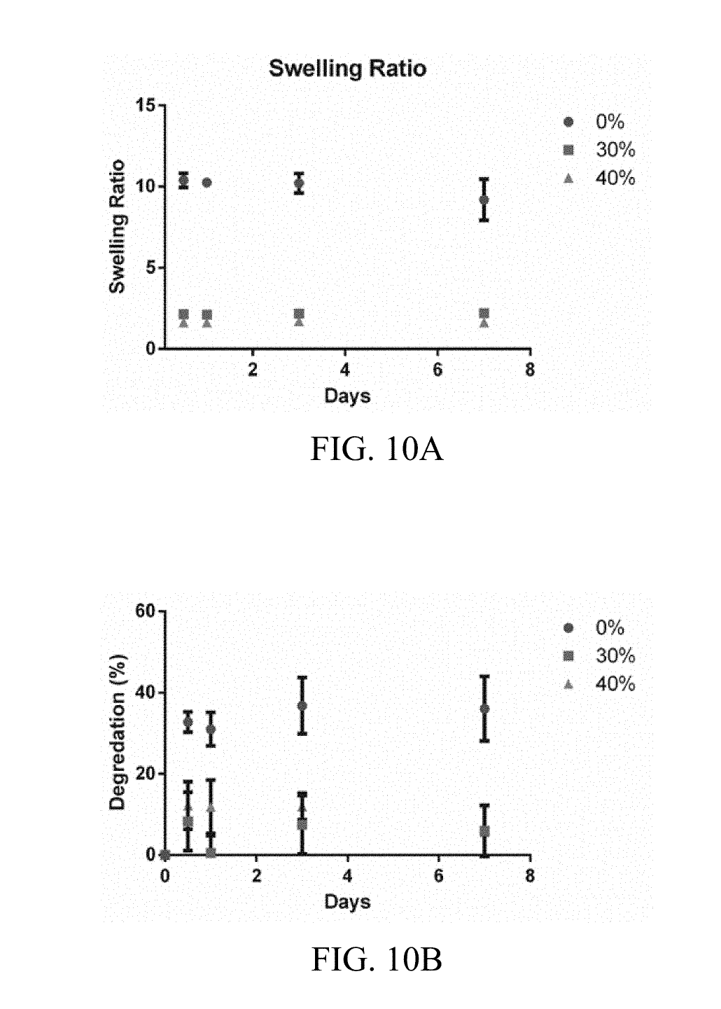

FIG. 10A illustrates the swelling ratio of three composite materials of the present invention with 0 wt. %, 30 wt. % and 40 wt. % HA whiskers. The swelling ratio of the material without the HA whiskers was higher compared to the swelling ratio of materials that included 30 or 40 wt. % of the HA whiskers.

FIG. 10B illustrated degradation of three composite materials of the present invention with 0 wt. %, 30 wt. % and 40 wt. % HA whiskers. The degradation was the highest for the materials that did not include HA whiskers.

### Example 5: Cellular Growth

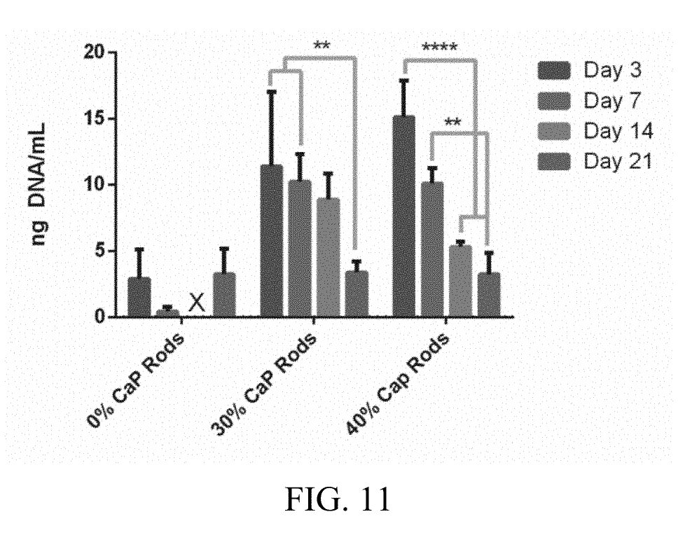

CaP-chitosan hydrogels were examined to determine if these materials had regenerative properties. FIG. 11 illustrates the relative number of cells for three composite materials of the present invention with 0 wt. %, 30 wt. % and 40 wt. % CaP rods. The deviation for the materials is significantly different and denoted with a ** (p<0.01) and **** (p<0.0001). The DNA was quantified to determine the relative number of cells over a three week period for these samples. Regenerative capacity is an essential feature of materials engineered in order to direct the regeneration of living tissues.

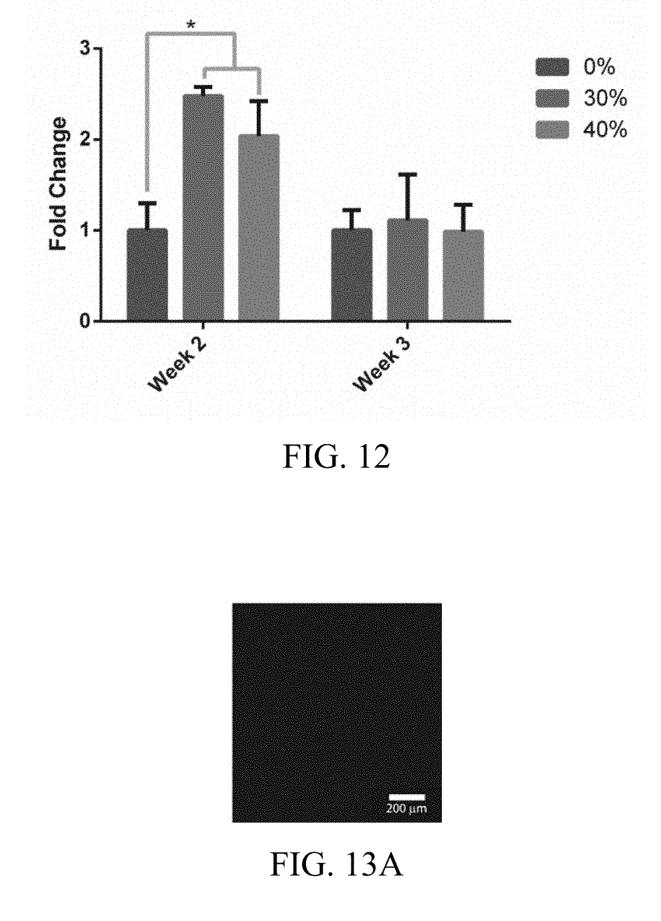

FIG. 12 illustrates the relative expression of RUNX2 on each of the HA composite materials for 0 wt. %, 30 wt. % and 40 wt. % HA whisker composite materials at 2 and 3 weeks. FIG. 12 expresses the fold change using 0% as the control. Two weeks illustrates the highest fold change, which then approaches the control after three weeks.

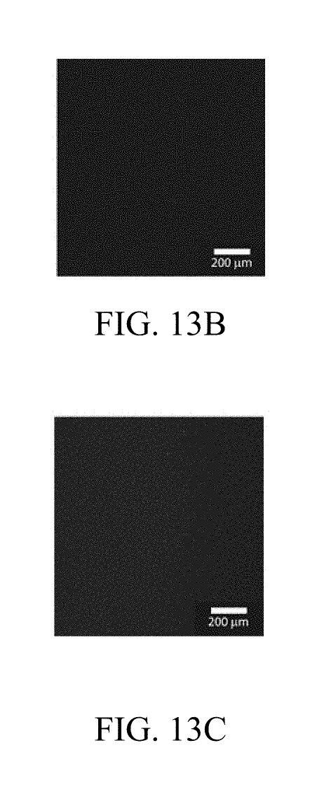

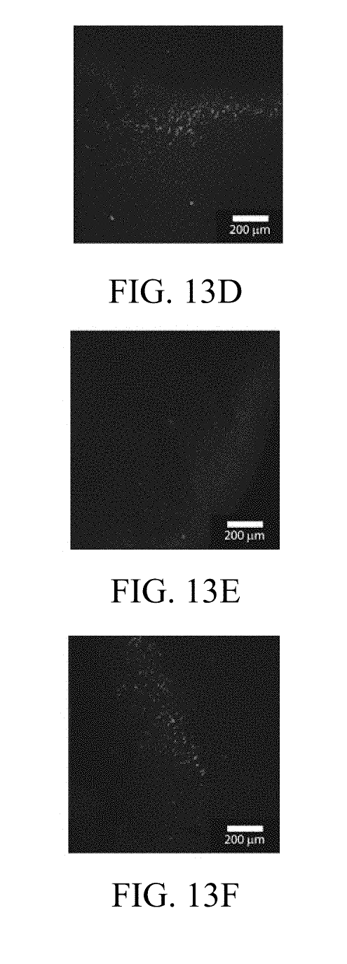

FIGS. 13A-13F illustrate confocal micrographs of dental pulp stem cells for various amounts of HA whiskers after 14 or 21 days. Cells were stained for osteocalin (pink) and nuclei were stained with DAPI (blue). Observation of cellular growth on all substrates indicates that composition of the hydrogels does not create an environment that is toxic towards cells and also supports cellular growth. Cellular growth is observed to be preferential on materials with incorporated CaP nanorods. Lack of cellular growth on the polymer-only (FIGS. 13A and 13B—no rods) substrate demonstrates that dental pulp stem cells do not have high affinity for the polymer alone, whereas the growth on CaP materials is as much as 3× more effective, indicating that incorporation of calcium phosphates to form a composite material is advantageous and important for regeneration as the CaP improves cell interaction and overall bioactivity of the system. Thus, CaP provides dual functionality by improving both mechanical strength and biological activity. Other configurations of CaP

Examples 6-9 illustrate examples where the CaP is not a rod, and can be a material other than HA alone.

### Example 6: TEM

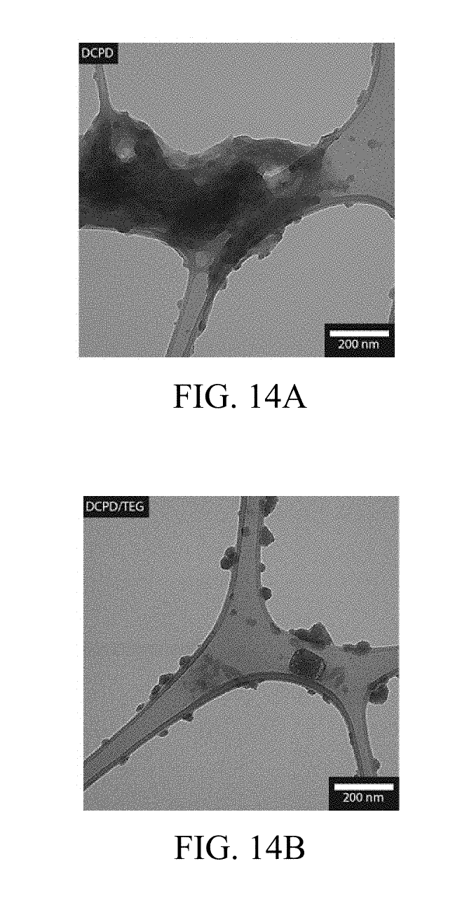

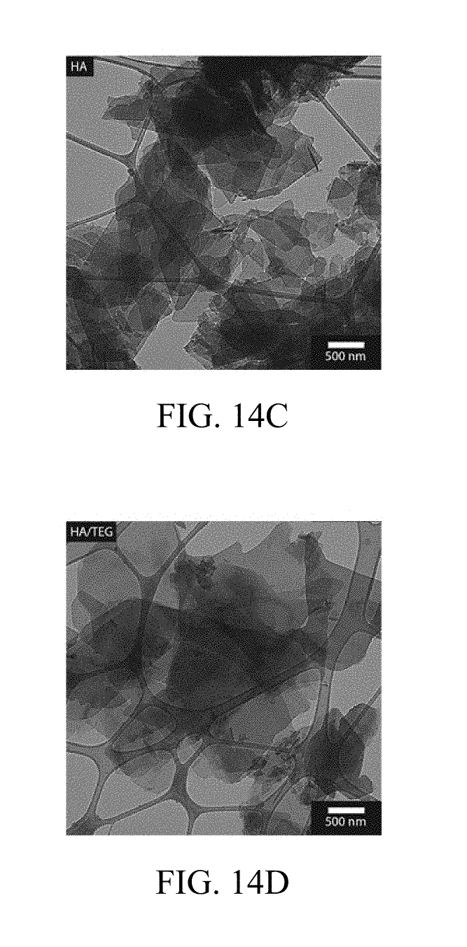

FIGS. 14A-14D illustrate other configurations of CaP. FIG. 14A illustrates DCPD, which is illustrated as an agglomerate in the polymer. As is illustrated in FIG. 14B, TEG can minimize the agglomeration of DCPD. Similarly, FIG. 14C illustrates HA plate particulates, which are agglomerated, while FIG. 14 D illustrates HA particulates with TEG which again can minimize agglomeration of the CaP additive in the polymer.

### Example 7: Storage Modulus

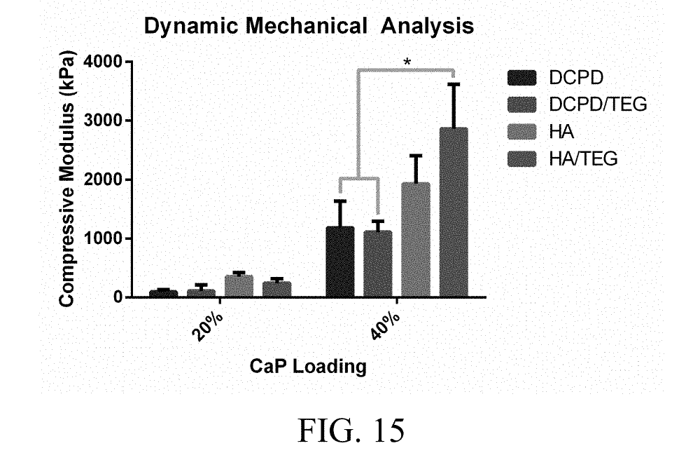

FIG. 15 illustrates the storage modulus for four different materials with various amounts of CaP of the present invention. FIG. 15 illustrates that the compressive modulus for HA/TEG in an amount of 40 wt. % is higher compared to HA/TEG that included 20 wt. % of this CaP.

### Example 8: Cellular Growth

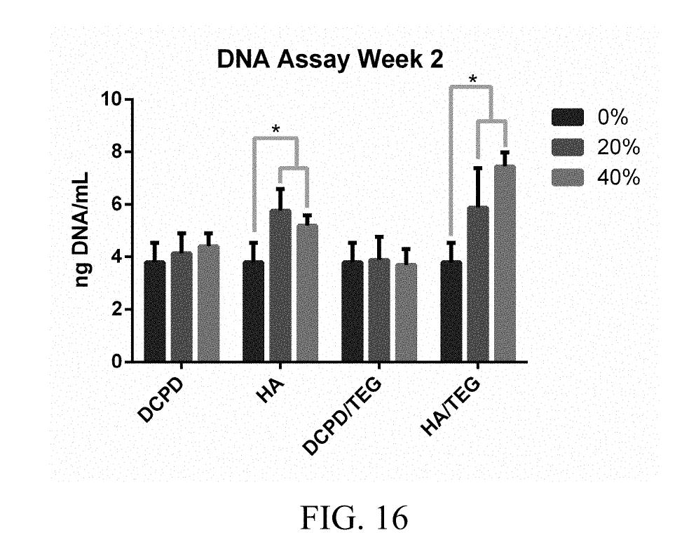

FIG. 16 illustrates the relative number of dental pulp stem cells over a 3 week period for the 0 wt. %, 20 wt. % and 40 wt. % loaded with various materials. DNA was quantified to determine the relative number of dental pulp stem cells over a three week period for the 0 wt. %, 20 wt. % and 40 wt. % loading of the four composites.

### Example 9: BSA Release

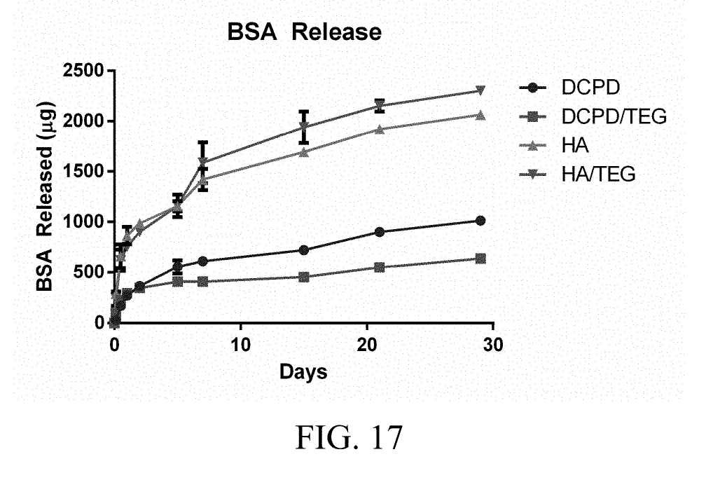

FIG. 17 illustrates BSA release over time for various compositions of the present invention. The BSA release was highest for HA/TEG over the period of time.

Ranges have been discussed and used within the forgoing description. One skilled in the art would understand that any sub-range within the stated range would be suitable, as would any number within the broad range, without deviating from the invention.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

1. A method of forming a hydrogel, comprising:
providing a modified polysaccharide, wherein the modified polysaccharide comprises a chitosan modified to form a soluble chitosan; and
crosslinking the modified polysaccharide with a diepoxide to form the hydrogel.

2. The method of claim 1, further comprising mixing between about 0 wt. % and about 40 wt. % total of at least one additive with the modified polysaccharide.

3. The method of claim 1, wherein the diepoxide is 1,4 butanediol diglycidyl.

4. The method of claim 1, wherein the soluble chitosan comprises a carboxymethyl group.

5. The method of claim 1, wherein the additive is at least one of a calcium phosphate, growth factors, chemokines, SDF-1a, and a bovine serum albumin.

6. The method of claim 5, wherein additive comprises the calcium phosphate and the calcium phosphate is at least one of a hydroxyapatite, a dicalcium phosphate dihydrate, a tetraethylene glycol diacrylate modified hydroxyapatite or a tetraethylene glycol diacrylate modified dicalcium phosphate.

7. The method of claim 6, wherein the calcium phosphate is the tetraethylene glycol diacrylate modified hydroxyapatite or the tetraethylene glycol diacrylate modified dicalcium phosphate, and wherein the tetraethylene glycol diacrylate is in excess.

8. A method to form an implantable material, comprising:
providing between about 2 wt. % and about 15 wt. % of a modified polysaccharide, wherein the modified polysaccharide comprises a polysaccharide modified with a carboxymethyl; and
providing between about 5 wt. % and about 20 wt. % of an diepoxide;
providing between about 0 wt. % and about 40 wt. % of a calcium phosphate; and
mixing the polysaccharide, the diepoxide and the calcium phosphate to form the implantable material.

9. The method of claim 8, wherein the diepoxide is 1, 4 butanediol diglycidyl ether.

10. The method of claim 8, wherein the calcium phosphate is at least one of a hydroxyapatite, dicalcium phosphate dihydrate, tetraethylene glycol diacrylate modified hydroxyapatite or tetraethylene glycol diacrylate modified dicalcium phosphate dihydrate.

11. The method of claim 8, wherein the polysaccharide is a chitosan.

12. The method of claim 8, further comprising providing between about 0 wt. % and about 40 wt. % of at least one additive to the modified polysaccharide.

13. The method of claim 8, further comprising curing the implantable material for between about 10 minutes and about 40 minutes to form a cured implantable material.

14. The method of claim 12, wherein the curing does not use equipment.

15. The method of claim 12, wherein the implantable material is self-curing.

16. The method of claim 11, wherein the additive is at least one of growth factor, chemokine, SDF-1a, or a bovine serum albumin.

17. A void filling gel material comprising a modified polysaccharide crosslinked with a diepoxide, wherein the gel hardens to form an implantable material.

18. The gel of claim 16, further comprising at least one additive.

19. The gel of claim 17, wherein the at least one additive is a calcium phosphate, growth factor, chemokine, SDF-1a, or a bovine serum albumin.

20. The gel of claim 17, wherein the calcium phosphate is at least one of a hydroxyapatite, a dicalcium phosphate, a tetraethylene glycol diacrylate modified hydroxyapatite or a tetraethylene glycol diacrylate modified dicalcium phosphate dihydrate.
